(19) United States
(12) Patent Application Publication
(10) Pub. No.: US 2016/0338305 A1
(43) Pub. Date: Nov. 24, 2016

(54) Ulmus L. plant named 'Guanyin'

(71) Applicant: Jianguo Wang, Pingdingshan (CN)

(72) Inventor: Jianguo Wang, Pingdingshan (CN)

(21) Appl. No.: 14/545,529

(22) Filed: May 18, 2015

(51) Int. Cl. A01H 5/12

(52) U.S. Cl. CPC A01H 5/12

(57) ABSTRACT

‘Guanyin’ is a distinctive variety of Ulmus L. Plant, which is characterized by its appearance and color of the whole plant.

### BACKGROUND OF THE INVENTION

The present invention relates to a new and distinct variety of Ulmus L. plant, which is given the varietal denomination of ‘Guanyin’.

The new Guanyin discovered by the applicant differs from related plants including Ulmus chinensis.

### SUMMARY OF THE NEW VARIETY

‘Guanyin’ is a distinctive variety of Ulmus L. plant, which is characterized by that new leaves in early spring are light yellow while a few new leaves have yellow-green spots; leaves in spring and summer have yellow-green spots; and leaves in summer and autumn have white-green spots.

### BRIEF DESCRIPTION OF THE DRAWINGS

FIG. 1 is a color photograph of the new invention, ‘Guanyin’, showing the whole plant with green leaves.

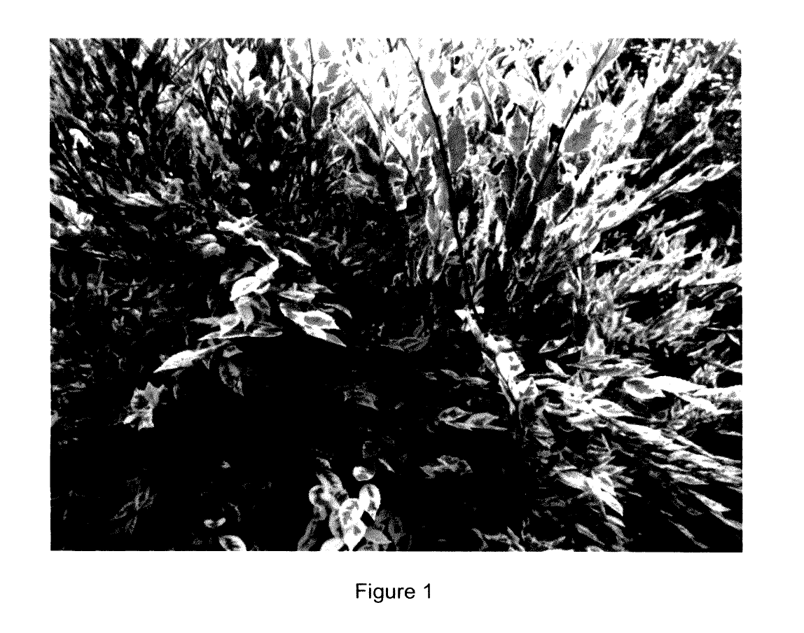

FIG. 2 is a color photograph of the new invention, ‘Guanyin’, showing the whole plant with white leaves.

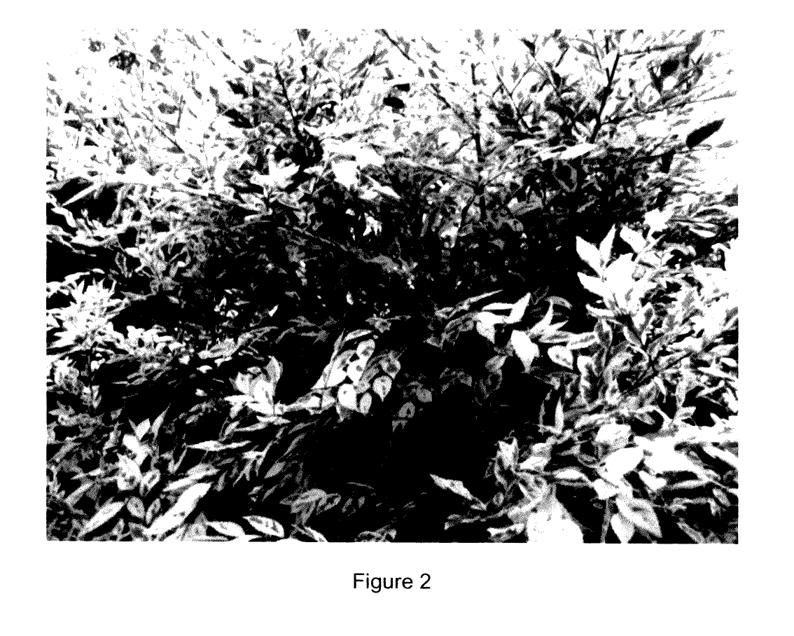

### DETAILED DESCRIPTION

The accompanying colored photographs illustrate the overall appearance of the new and distinct variety of the Ulmus L. plant, which is known as ‘Guanyin’. The descriptions disclosed herein are based upon observations of the plant growth.

‘Guanyin’ buds a week earlier than Ulmus chinensis in spring. New leaves thereof are light yellow, and drop after about 20 days. Reborn leaves are gray-green with white edges. After about 30 days, outer leaves grow light yellow spots. With sunlight becoming strong, the leaves are changed from light yellow to yellow-white. From summer to autumn, outer mature leaves have light white spots, new leaves have light yellow or yellow-white spots, and inner leaves are gray-green with white edges.

The ‘Guanyin’ has small leaves, which is ½ of a size of Ulmus pumila L. leaves. An amount of the leaves are doubled to the one of Ulmus pumila L. leaves. In winter, top buds of the ‘Guanyin’ wither while many side buds grow. Branches are slim and close to each other, and branch capability is strong. A crown is egg-shaped and compact. The ‘Guanyin’ inherits characteristics of a female parent Ulmus chinensis such as cold tolerance, drought tolerance and salt tolerance. After ten years of observation and 30000 strains of asexual reproduction, the variety has sufficient stability and consistency.

The variety has cold tolerance, drought tolerance, salt tolerance and shade tolerance, and is able to grow in most parts of the northern hemisphere. The variety is suitable for rootstock asexual reproduction with Ulmus chinensis, Ulmus laevis and Ulmus americana. The variety is suitable for grafting on Ulmus chinensis var. pendula and Ulmus laevis var. pendula, into a shape of a flower, without growth imbalance. The variety is able to be cultivated as foliage plants and bonsai.

| similar variety | | feature of similar variety | feature of the New Variety |
|---|---|---|---|
| 1 | Ulmus pumila | leaves are always green | new leaves in early spring are |
| | | during growth | light yellow while a few new |
| | | | leaves have yellow-green |
| | | | spots; leaves in spring and |
| | | | summer have yellow-green |
| | | | spots; and leaves in summer |
| | | | and autumn have white-green |
| | | | spots |
| | | leaves are large | leaves are small |
| 2 | | branches are far from | branches are slim and close to |
| | | each other | each other |

These features and other characteristics of the plant are apparent from the figures.

What is claimed is:

1. A new and distinct variety of plant named ‘Guanyin’, substantially as described and illustrated, classified in the family Ulmus L. and characterized by that leaves in early spring are light yellow; leaves in spring and summer have yellow-green spots; and leaves in summer and autumn have white-green spots.
